SEQUENCE LISTING (2 sequences)

SEQ ID NO: 1 - protein, 22 aa, Homo sapiens
CKASAFGKDHPRYYATYINKDP

SEQ ID NO: 2 - protein, 157 aa, Homo sapiens
SHRDTASHVQSSPVQSDSSTAKEHGHFSSLSQDSAYHSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

(19) United States
(12) Patent Application Publication
(10) Pub. No.: US 2019/0079080 A1
(43) Pub. Date: Mar. 14, 2019

(54) ANTIBODY SPECIFIC TO PROFILAGGRIN C-TERMINAL DOMAIN, AND USE THEREOF

(71) Applicant: Shiseido Company, Ltd., Tokyo (JP)

(72) Inventors: Toshihiko HIBINO, Yokohama-shi, (JP); Mami YAMAMOTO, Tokyo (JP); Masaya TAKAGI, Yokohama-shi (JP); Junichi SAKABE, Hamamatsu-shi (JP); Yoshiki TOKURA, Hamamatsu-shi (JP)

(73) Assignee: Shiseido Company, Ltd., Tokyo (JP)

(21) Appl. No.: 16/136,929

(22) Filed: Sep. 20, 2018

Related U.S. Application Data
(62) Division of application No. 14/356,985, filed on May 8, 2014, now abandoned.

(30) Foreign Application Priority Data
Nov. 9, 2011 (JP) 2011-245483

(51) Int. Cl. G01N 33/53; C07K 16/18; G01N 33/68

(52) U.S. Cl. CPC G01N 33/53; C07K 16/18; G01N 2800/202; G01N 33/6881

(57) ABSTRACT

The present invention provides an antibody specific to a C-terminal domain of a human profilaggrin gene, wherein the C-terminal domain is a peptide comprising the amino acid sequence set forth in SEQ ID NO:1 or a variant thereof, a method for detecting a filaggrin gene mutation using the antibody, and a kit for the detection.

### CROSS-REFERENCE TO RELATED APPLICATIONS

This application is a Divisional of U.S. application Ser. No. 14/356,985, which is the U.S. National Stage application of PCT/JP2012/079015, filed Nov. 8, 2012, which claims priority from Japanese application JP 2011-245483, filed Sep. 11, 2011.

The instant application contains a Sequence Listing which has been submitted in ASCII format via EFS-WEB and is hereby incorporated by reference in its entirety. Said ASCII copy, created on Sep. 19, 2018, is named sequence.txt and is 2.

### TECHNICAL FIELD

The present invention provides an antibody specific to a C-terminal domain of profilaggrin and a use thereof, specifically, a method for detecting a mutation in a filaggrin gene using the antibody, and a kit for the detection.

### BACKGROUND ART

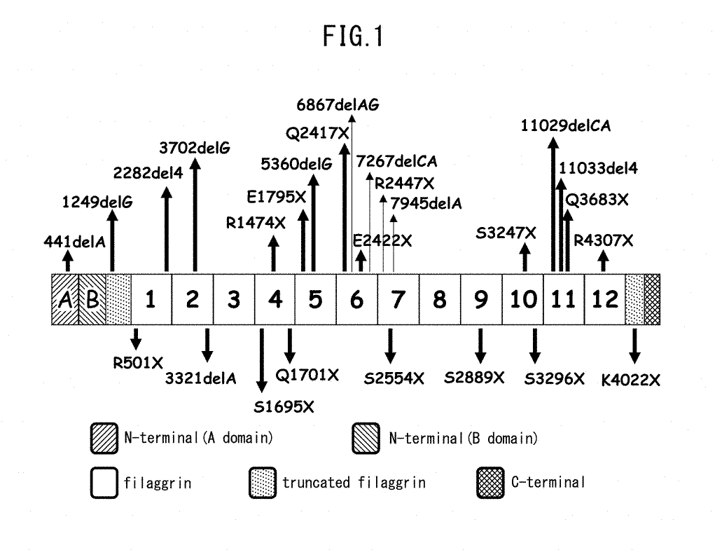

Keratin fibers of the granular layer of the skin bind to a protein referred to as filaggrin and aggregate during keratinization, and form a unique pattern referred to as a “keratin pattern”. A precursor substance of filaggrin is referred to as profilaggrin and is stored in keratohyalin granules within granular cells. The profilaggrin has a structure comprising filaggrin repeats consisting of 10 to 12 continuous filaggrin molecules interposed between the N-terminal domain and the C-terminal domain (FIG. 1). Along with keratinization, these domains, etc., are cut out and individual filaggrin molecules are produced. When the filaggrin finally degrades, a natural moisturizing factor (NMF) is formed. The constituent components of NMF are amino acids and their derivatives, which are known to play an important role in maintaining the moisture of the skin (Horii I, Kawasaki K, Koyama J et al. J Dermatol. 1983; 10: 25-33).

Mutations in filaggrin cause a reduction of NMF, and as a result, cause a decrease in the water-retaining capability of the horny layer. However, recent research has indicated that a genetic mutation of filaggrin is the cause of ichthyosis and some atopic dermatitis (Sandilands A, Terron-Kwiatkowski A, Hull P R, et al. Nat Genet 2007; 39: 650 to 654), surprisingly revealing that the mutations in filaggrin not only reduce the water-retaining capability of the horny layer resulting from the decrease in NMF, but also directly influence a barrier function.

The aforementioned diseases result from the abnormalities in the barrier function of the horny layer, as described in the guidelines for the treatment of atopic dermatitis reported in the Journal of the Japan Dermatological Association in 2010 (Norito Kato, “Treatment Guidelines and New Treatments for Atopic Dermatitis”, Journal of the Japanese Dermatological Association 2010, 120(13), 2564-2565), which states that “the pathological conditions of atopic dermatitis accompany a physiological abnormality of the skin, such as dry skin and barrier function abnormality resulting from the abnormalities in the epidermis, specifically the horny layer”.

Subsequent research has estimated that approximately half of atopic dermatitis in Europe and approximately 30% of atopic dermatitis in Japan result from genetic mutations of filaggrin. From this point, it is sufficiently understood that atopic dermatitis is a multifactorial disease. However, it is critically important to judge the presence or absence of a mutation in the filaggrin gene for decision on courses of future treatment. Further, regarding a test subject free of atopic dermatitis, it will become possible to predict that the subject is highly likely to suffer from atopic dermatitis in the future if a mutation in a filaggrin gene is detected.

However, numerous mutations in the filaggrin gene have been reported. Specifically, such mutations do not exist in a specific region, but exist almost over the entirety (FIG. 1). Further, it has been reported that filaggrin repeats are unlikely to be produced if there is a mutation in the filaggrin gene itself or in a part of the C-terminal region of a gene encoding profilaggrin, which is a precursor of filaggrin (Sandilands A, Terron-Kwiatkowski A, Hull P R et al. Nat Genet 2007; 39: 650-654). Therefore, in order to specify whether or not there is a mutation in filaggrin, it is necessary to sequence the entire profilaggrin gene. However, even current genetic analysis technology cannot easily analyze the profilaggrin gene.

For example, filaggrin repeats, which are the most important region in the profilaggrin gene, are slightly different from each other, and all the filaggrin repeats must be sequenced accurately. Further, the mutations in a filaggrin gene vary depending on an ethnic group. As a result, it is remarkably difficult to determine that a difference of one base is a mutation, due to the variation in the various individual repeats in these various regions, and moreover, due to the ethnic variation. Therefore, currently, mutations in a profilaggrin gene can be analyzed in only specific facilities in the world.

### PRIOR ART DOCUMENTS

### Non-Patent Literature

Non-patent literature 1: Horii I, Kawasaki K, Koyama J, et al, J Dermatol. 1983: 10: 25-33
Non-patent literature 2: Sandilands A, Terron-Kwiatkowski A, Hull P R, et al. Nat Genet 2007; 39: 650-654
Non-patent literature 3: Kato, Norito “Treatment Guidelines and New Treatments for Atopic Dermatitis”, Journal of the Japanese Dermatological Association 2010, 120(13), 2564-2565

### SUMMARY OF THE INVENTION

### Problems to be Solved by the Invention

The object of the present invention is to provide a new antibody specific to a C-terminal domain of profilaggrin, and a method for detecting a mutation in a filaggrin gene using the antibody, and a kit for the detection.

### Means for Solving the Problems

The present inventors have discovered that a filaggrin gene mutation can be detected non-invasively and simply, using an antibody specific to the C-terminal domain, and have completed the present invention.

Therefore, the present application includes the following inventions:
(1) An antibody specific to a C-terminal domain of a human profilaggrin gene, wherein the C-terminal domain is a peptide comprising the amino acid sequence set forth in SEQ ID NO:1 or a peptide comprising an amino acid sequence derived from the amino acid sequence set forth in SEQ ID NO:1 by deletion, substitution, or addition of one or more amino acids.
(2) A method for detecting a mutation in a filaggrin gene, comprising the steps of:
detecting the presence or absence of said C-terminal domain in a skin sample of a subject using the antibody of (1), and
judging that the mutation in a filaggrin gene is present in the subject, in case when said C-terminal domain is not detected.
(3) The method according to (2), wherein the method comprises judging that the subject is suffering or is highly likely to suffer from atopic dermatitis, in case when said C-terminal domain is not detected.
(4) The method according to (3), wherein said skin sample is a horny layer sample obtained by tape stripping.
(5) A kit comprising the antibody according to (1) for detecting a mutation in a filaggrin gene.

### Effect of the Invention

If a mutation is generated in a profilaggrin gene, filaggrin repeats following the mutation, and eventually, the C-terminal domain of the profilaggrin cannot be created. According to the present invention, the presence or absence of the C-terminal domain within the skin sample can be detected using an antibody specific to the domain to simply detect the presence or absence of a filaggrin gene mutation. Further, a profilaggrin gene mutation may be a cause of atopic dermatitis, and thus, the diagnosis or prediction of atopic dermatitis using the antibody of the present invention can be performed non-invasively and simply.

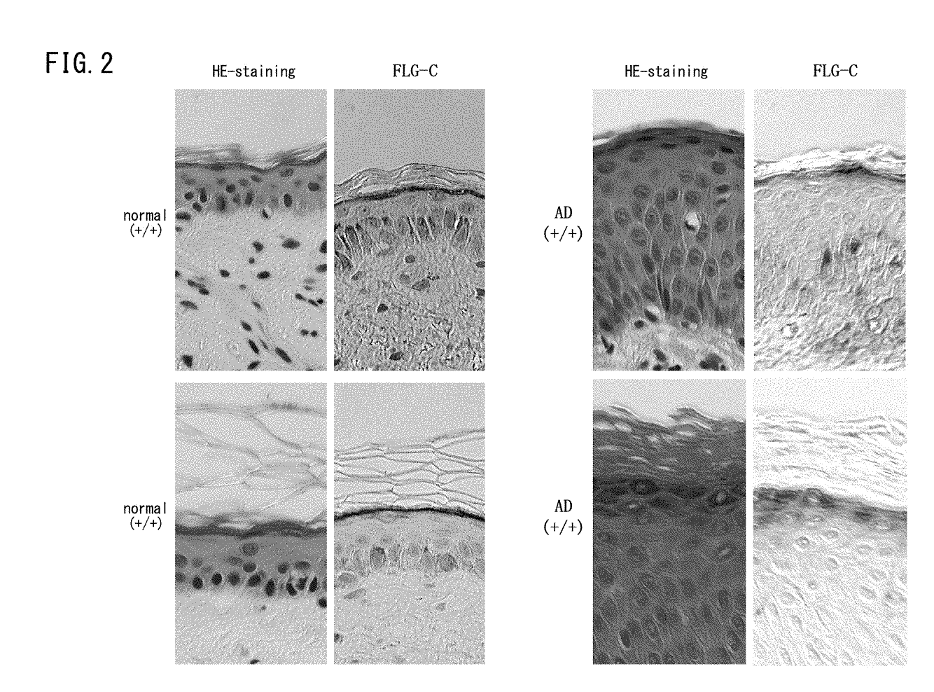

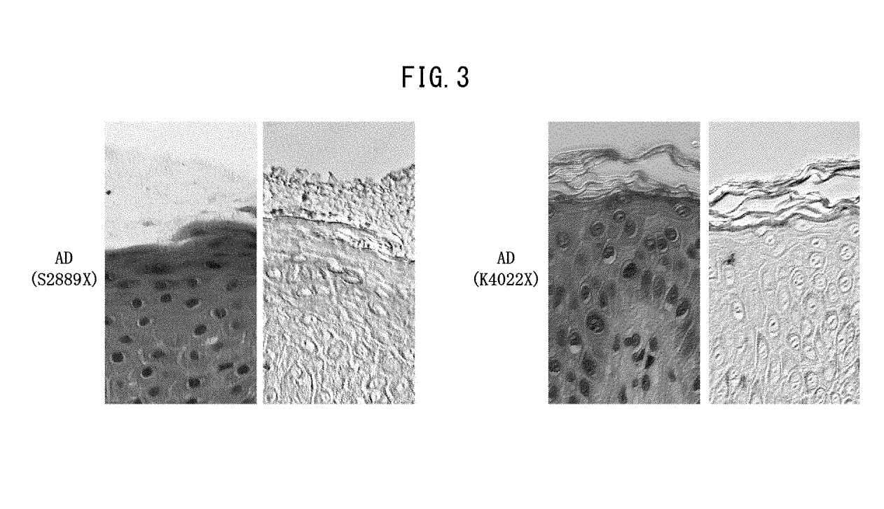

### BRIEF DESCRIPTION OF THE DRAWINGS

[FIG. 1] is a schematic illustration showing a structure of the profilaggrin gene (Chrlq21.3/Human) having 12 filaggrin repeats, and mutation sites of the gene.

[FIG. 2] is an immunostaining diagram of normal skin (normal (+/+)) without any profilaggrin gene mutations and of atopic dermatitis skin (AD(+/+)). The drawings on the left side show the results of staining with Hematoxylin-Eosin (HE staining), and the drawings on the right side show the results of staining with an antibody specific to the C-terminal domain of profilaggrin and a chromogenic substrate DAB (3,3′-diaminobenzidine) (FLG-C).

[FIG. 3] is an immunostaining diagram of atopic dermatitis skin with profilaggrin gene mutations (S2889X; K4022X). The drawings on the left side show the results of staining with Hematoxylin-Eosin (HE staining), and the drawings on the right side show the results of staining with the antibody specific to the C-terminal domain of profilaggrin and a chromogenic substrate DAB (3,3′-diaminobenzidine) (FLG-C).

### EMBODIMENT FOR CARRYING OUT THE INVENTION

According to a first aspect of the present invention, there is provided an antibody specific to a C-terminal domain of a human profilaggrin gene, wherein the C-terminal domain is a peptide comprising the amino acid sequence set forth in SEQ ID NO:1 or a variant thereof. Filaggrin is roughly categorized into Type I and Type II. However, the term “profilaggrin” as used herein refers to Type I. Profilaggrin has a structure comprising filaggrin repeats consisting of 10 to 12 continuous filaggrin molecules interposed between the N-terminal domain and the C-terminal domain (FIG. 1). SEQ ID NO:2 indicates the entire sequence of a C-terminal domain consisting of 157 amino acids.

With respect to the C-terminal domain, the amino acid sequence set forth in SEQ ID NO:1 is not homologous to either Type II profilaggrin or hornerin, which is a profilaggrin-analogous molecule, and is a sequence specific to Type I. Therefore, an antibody specific to the amino acid sequence does not cross-react with these analogous proteins.

The variant may be any variant as long as an antibody obtained using the variant as an antigen does not recognize a peptide having a sequence analogous to the amino acid sequence set forth in SEQ ID NO:1 in the skin and is specific to the C-terminal domain of a human profilaggrin gene. For example, the variant may be a peptide comprising an amino acid sequence derived from the amino acid sequence set forth in SEQ ID NO:1 by deletion, substitution, or addition of one or more amino acids.

While not limited thereto, the peptide can be obtained by chemically synthesis according to a conventional method. The antibody of the present invention can be obtained by administering the peptide as the antigen, preferably an antigen complex comprising the peptide bound to a suitable carrier, to mammals, for example, rats, mice, rabbits, etc. While not limited thereto, the dosage amount of the antigen or antigen complex per animal is 0.1 to 100 mg when no adjuvant is used, and is 10 to 1000 μg when an adjuvant is used. Examples of the adjuvant include Freund's Complete Adjuvant (FCA), Freund's Incomplete Adjuvant (FIA), aluminum hydroxide adjuvant, etc.

The immunization is mainly performed by intravenous, subcutaneous, or intraperitoneal injection. Further, the immunization interval is not specifically limited. However, immunization is performed 1 to 10 times, preferably 2 to 5 times, at an interval of several days to several weeks, preferably, an interval of 2 to 5 weeks. Moreover, the antibody titer is measured by Western blotting, Enzyme-Linked Immunosorbent Assay (ELISA or EIA), Radioimmunoassay (RIA), etc., 6 to 60 days after the day of the final immunization. Preferably, blood is collected and antiserum is obtained on the day when the maximum antibody titer is exhibited.

The polyclonal antibody may be purified by affinity chromatography using a column to which the antigen peptides are bound, and other purification methods known to a person skilled in the art, for example, ion exchange chromatography, gel electrophoresis chromatography, high-performance liquid chromatography, etc. Further, the antibody of the present invention may be a monoclonal antibody. The monoclonal antibody may be prepared according to an already known method using a peptide having the amino acid sequence set forth in SEQ ID NO:1 or a variant thereof.

According to a second aspect of the present invention, there is provided a method for detecting a mutation in a filaggrin gene, wherein the method comprises the steps of: detecting the presence or absence of said C-terminal domain in a skin sample of a subject with the antibody, and judging that the mutation in a filaggrin gene is present in the subject, in case when said C-terminal domain is not detected. The term “a mutation in a filaggrin gene” as used herein refers to one or more mutations of a filaggrin gene, which inhibit the formation of the C-terminal domain of profilaggrin. Examples of the mutations include the mutation S2889X in which termination occurs at the serine in position 2889, the mutation K4022X in which termination occurs at the lysine in position 4022, etc. (FIG. 1).

A mutation in the profilaggrin gene leads to atopic dermatitis. Therefore, according to the method of the present invention, when the C-terminal domain is not detected, it is judged that the subject is suffering or is highly likely to suffer from atopic dermatitis.

The detection of the presence or absence of the C-terminal domain is carried out by an immunological method well known to a person skilled in the art, for example, immunoassay (ELISA, RIA, etc.) The skin samples are collected by any method from the arms, legs, head, etc., of subjects. The subjects are mammals, preferably humans with suspected skin diseases caused by filaggrin gene mutations, specifically, atopic dermatitis. The method for collecting the skin samples is not specifically limited, but a tape stripping method is preferable because it is simple and non-invasive. The tape stripping is a method for collecting a horny layer sample, which comprises bonding a piece of adhesive tape to the skin surface layer and peeling off the piece so that the skin horny layer adheres to the peeled adhesive tape. According to the tape stripping method, a skin sample containing a profilaggrin gene can be simply obtained, and a filaggrin gene mutation can be detected non-invasively.

A preferable method of tape stripping is as follows: First, a surface layer of skin is cleaned with, for example, ethanol, etc., to remove sebum, dirt, and the like. A piece of adhesive tape cut to a suitable size (for example 5×5 cm) is gently placed on the skin surface. The entire piece of tape is pressed flat onto the skin surface by applying uniform pressure, and the adhesive tape is subsequently peeled off while applying uniform force. The adhesive tape may be a commercially available cellophane tape, for example, Scotch Superstrength Mailing Tape (manufactured by 3M), Cellophane Tape (CelloTape(R), Nichiban), etc.

A neutral to weakly alkaline buffer including a non-ionic surfactant can be used to extract a soluble component from the tape-stripped horny layer, and includes, but not limited to the following buffer: 100 mM Tris HCl+0.14 M NaCl+0.1% Triton ×100]. The tape having a horny layer adhering thereto is cut into a strip with scissors, etc., transferred to a container, such as an Eppendorf tube, immersed in a small amount of the aforementioned buffer, stirred by inversion, to efficiently extract the soluble components. The extracted soluble components are subjected to an immunoassay, etc., using the antibody of the present invention, to judge the presence or absence of a profilaggrin gene mutation.

According to a third aspect of the present invention, there is provided a kit for detecting a mutation in a filaggrin gene, comprising the antibody. The kit may comprise the antibody of the present invention in a container. The kit may further comprise reagents required for the aforementioned detection method, for example, in case of the detection of a mutation by the ELISA method, an enzyme conjugate, substrate solution, quenching solution, wash solution, enzyme conjugate diluent, assay buffer, control, sample diluent, conjugate, etc., and operating instructions explaining the procedures of the method.

The present invention will be specifically explained below in more detail with reference to specific examples. However, the present invention is not limited thereto.

### EXAMPLES

Immunohistochemical Staining

Skin section samples were prepared from normal skin and atopic dermatitis skin, which were free of a profilaggrin gene mutation, and atopic dermatitis skin with a profilaggrin gene mutation (S2889X or K422X). The immunohistochemical staining of the skin fragment was performed by the method described in Kamata et al. (J. Biol. Chem., Vol. 284, Issue 19, 12829-12836, May 8, 2009). The antibody specific to the profilaggrin C-terminal domain was obtained by immunizing a rabbit with a synthetic peptide having the amino acid sequence CKASAFGKDHPRYYATYINKDP (SEQ ID NO: 1) as the immunogen (manufactured by Sigma Inc.)

The normal skin and atopic dermatitis skin, which were free of a profilaggrin gene mutation, were stained with Hematoxylin-Eosin, or with the antibody specific to a profilaggrin C-terminal domain and a chromogenic substrate DAB (3,3′-diaminobenzidine). The results are shown in FIG. 2. Profilaggrin was localized in the granular layer. The color development of DAB staining in the granular layer was reduced in the atopic dermatitis skin free of a profilaggrin gene mutation compared to normal skin (refer to the immunostaining diagram of FLG-C). Namely, the results of FIG. 2 reveal that the expression of filaggrin is reduced in atopic dermatitis skin even free of a profilaggrin gene mutation.

Next, immunostaining was performed for atopic dermatitis skin with the profilaggrin gene mutation S2889X or K422X. In the same manner as the results in FIG. 2, each skin was stained with Hematoxylin-Eosin (left side: HE stain), or with the antibody specific to a profilaggrin C-terminal domain and a chromogenic substrate DAB (3,3′-diaminobenzidine) (right side: FLG-C), and the results are shown in FIG. 3. The granular layer was not stained with DAB unlike the results in FIG. 2. Namely, it was revealed that the profilaggrin C-terminal was not formed by the mutation in the atopic dermatitis skin with the mutation.

The aforementioned results have revealed that the antibody specific to the profilaggrin gene C-terminal domain of the present invention could be used to simply detect the presence or absence of filaggrin gene mutations known to be many, without verifying every mutation by sequencing, and thereby to judge that the skin of a subject is suffering or is highly likely to suffer from atopic dermatitis.

1-5. (canceled)

6. A method of detecting a mutation in a filaggrin gene, comprising the steps of:
obtaining a skin sample from a subject suffering from or suspected of having atopic dermatitis;
determining whether a C-terminal domain of profilaggrin is present or absent in the sample, wherein the presence of the C-terminal domain of profilaggrin in the sample indicates that the filaggrin gene is not mutated and the absence of the C-terminal domain of profilaggrin in the sample indicates that the filaggrin gene is mutated, and
subjecting the subject to a treatment or prevention of atopic dermatitis if being determined that the filaggrin gene is mutated.

7. The method of claim 6, wherein the C-terminal domain of profilaggrin comprises the amino acid sequence of SEQ ID NO: 1.

8. The method of claim 6, wherein the skin sample is obtained by tape stripping.

9. The method of claim 6, wherein the skin sample comprises a horny layer of the skin.

10. The method of claim 6, wherein determining whether the C-terminal domain of profilaggrin is present in the sample comprises performing an immunoassay.

11. The method of claim 10, wherein the immunoassay is an enzyme-linked immunosorbent assay (ELISA).

12. The method of claim 10, wherein the immunoassay is a radioimmunoassay (RIA).

13. The method of claim 10, wherein the immunoassay is a Western blot.

14. The method of claim 6, wherein determining whether the C-terminal domain of profilaggrin is present in the sample comprises immunostaining.

14. The method of claim 6, wherein the mutation in the filaggrin gene results in termination of a filaggrin protein at amino acid positions 2889 or 4022.

16. The method of claim 6, wherein an absence of the C-terminal domain of profilaggrin in the sample indicates the filaggrin gene is mutated.

17. The method of claim 6, wherein the subject is a human.

18. A method of detecting a mutation in a filaggrin gene, comprising the steps of
(a) obtaining a skin sample comprising a horny layer of the skin from a human subject,
(b) detecting a C-terminal domain of profilaggrin in the sample by contacting the sample with an antibody that binds to the C-terminal domain of profilaggrin; wherein an inability to detect the C-terminal domain of profilaggrin in the sample indicates the filaggrin gene is mutated, and
(c) subjecting the subject to a treatment or prevention of atopic dermatitis if being determined that the filaggrin gene is mutated.

19. The method of claim 18, wherein the human subject is suffering from or suspected of having atopic dermatitis.

20. The method of claim 18, wherein the C-terminal domain of profilaggrin comprises the amino acid sequence of SEQ ID NO: 1.

21. The method of claim 18, wherein the skin sample is obtained by tape stripping.

22. The method of claim 18, wherein contacting the sample with the antibody that binds to the C-terminal domain of profilaggrin is performed as part of an enzyme-linked immunosorbent assay (ELISA), a radioimmunoassay (RIA), a Western blot, or an immunostain.

23. The method of claim 18, wherein the mutation in the filaggrin gene results in termination of a filaggrin protein at amino acid positions 2889 or 4022.
